(19) United States
(12) Patent Application Publication
(10) Pub. No.: US 2014/0316302 A1
(43) Pub. Date: Oct. 23, 2014

(54) SAMPLE COLLECTION KIT

(71) Applicants: Sheena Nonnemacher, Bethlehem, PA (US); Kristen Blum, Whitehall, PA (US); Dean Fritch, Bally, PA (US); Mike Savard, Macungie, PA (US); Keith Kardos, Bethlehem, PA (US); Brian Murphy, Baltimore, MD (US); Jeremy Savage, Baltimore, MD (US); Erik Askin, Baltimore, MD (US)

(72) Inventors: Sheena Nonnemacher, Bethlehem, PA (US); Kristen Blum, Whitehall, PA (US); Dean Fritch, Bally, PA (US); Mike Savard, Macungie, PA (US); Keith Kardos, Bethlehem, PA (US); Brian Murphy, Baltimore, MD (US); Jeremy Savage, Baltimore, MD (US); Erik Askin, Baltimore, MD (US)

(73) Assignee: ORASURE TECHNOLOGIES, INC., Bethlehem, PA (US)

(21) Appl. No.: 14/131,726

(22) PCT Filed: Jul. 16, 2012
(86) PCT No.: PCT/US12/46879

§ 371 Date: Jun. 19, 2014

Related U.S. Application Data
(60) Provisional application No. 61/508,299, filed on Jul. 15, 2011.

(51) Int. Cl. A61B 10/00

(52) U.S. Cl. CPC A61B 10/0096; A61B 10/0051
USPC 600/582

(57) ABSTRACT

A sample collection kit includes a sample collector, a polycarbonate container, and a preservative solution. The sample collector includes an absorbent pad with a collecting element that receives a sample of oral fluid. The absorbent pad also includes an interior portion that extends into a handle. The handle includes an upper casing and a lower casing. The collecting element can be treated with a surfactant to optimize recovery of analytes from the oral sample and/or their absorbance onto the absorbent pad. A polycarbonate container receives the absorbent pad or the sample collector and stores it for testing of analytes. A dextran sulfate preservative solution is included in the polycarbonate container that inhibits enzymatic activity on a collected sample.

### TECHNICAL FIELD

The invention relates generally to a device, method, and system for collecting samples of bodily fluids. More particularly, the invention also relates to an oral-fluid collecting device and kit for collection and testing of the fluid.

### BACKGROUND

Humans produce up to 1.5 liters of saliva each day. The use of saliva or “oral fluid” samples is well established for substance of abuse or drug testing and disease testing. Collecting oral fluid specimens is generally considered to be less invasive and less embarrassing, and less stigmatizing than the collection of other bodily fluids, such as blood, serum, urine, etc. The term “oral fluid” is generally considered a better descriptor than “saliva” for the fluid collected in oral specimens. Oral fluids are produced from multiple glands in the mouth. Oral fluid is made up of both saliva and mucosal excretions. Oral fluids contain glandular and cellular debris present in the oral cavity as well as components of blood which include antibodies and drug metabolites.

Oral fluid samples may be collected in a number of ways in order to test for the presence of analytes. One type of sample collector typically includes an absorbent pad for absorbing the target fluid and a holder for holding the sample as the sample is being collected. Once the sample is absorbed by the absorbent pad, the entire pad is transferred to a vial. The vial is then delivered for testing. Disadvantageously, these systems still require additional manipulation, such as centrifugation of the sample in the vial, before the sample can be tested. Other types of sample collectors may release, or express, the sample from the absorbent pad into the vial, rather than placing the entire pad in the vial. Alternatively, the sample may be introduced directly into a testing device, such as a lateral test flow device, rather than storing the sample in a vial for subsequent testing. In particular, with typical devices, a precise quantity of oral fluid is not delivered. A metered quantity of oral fluid is critical to ensure that the quantity is sufficient for testing purposes and to allow determination of actual oral fluid concentrations when the oral fluid is combined with a preservative solution.

### SUMMARY

Example embodiments of the claimed invention provide a system, device, and method for improved collection and expression of oral fluids. For example, a number of embodiments enable users to manipulate the collection device to release an appropriate volume of sample fluid, which can be tested. Moreover, embodiments facilitate sample processing at the testing site of the sample fluid, which is stored and delivered in a vial. For instance, centrifugation can be eliminated as a necessary processing step.

One example embodiment of the claimed invention is an oral fluid sample collection kit that includes a sample collector, a polycarbonate container, and a preservative solution in the polycarbonate container. The sample collector includes an absorbent pad with a collecting element and an interior portion and a handle. The handle includes an upper casing and a lower casing. The absorbent pad extends from the collecting element into the handle. The polycarbonate container receives the absorbent pad or the sample collector itself and holds the pad or collector until testing occurs. The preservative solution in the container inhibits enzymatic activity on a collected sample that can otherwise destroy analytes of interest or otherwise alter the concentration of the analytes for which testing is being conducted. The preservative solution can be a dextran sulfate preservative solution, for example, that inhibits enzymatic activity on the collected sample.

One example embodiment of the claimed oral fluid sample collection kit includes a handle with a sample adequacy window. The sample adequacy window is positioned in the handle and provides visual access to the absorbent pad, which extends beyond the sample adequacy window. The sample adequacy window provides a visual indication as to the sufficiency of the amount of oral fluid sample that is collected. Additionally, the interior portion of the absorbent pad can include an indicator dye positioned between the junction of the collecting element and the interior portion and the sample adequacy window in the handle.

An example embodiment of the claimed invention can include a handle that includes internal pins positioned to fit and secure the upper casing and the lower casing together to form the handle. Additionally, the handle can include textured ridges arranged on the upper casing and/or the lower casing to provide secure handling of the sample collector.

In one embodiment, the collecting element of the absorbent pad can be treated with a hypertonic salt solution including 80 to 170 mg/mL NaCl and 14.3 mg/mL citrate buffer as well as a flavorant or sweetener.

The oral fluid sample collection kit of the claimed invention can also include a preservative solution in the container. The preservative solution can include an anti-microbial agent, an antibacterial agent, an anti-fungal agent, a bacteriostatic agent, a fungistatic agent, an enzyme inhibitor, and combinations of these agents.

In addition to the claimed sample collecting kit, one embodiment of the invention includes a method of collecting an oral fluid sample. The method of the claimed invention includes receiving an oral fluid sample with a sample collector, receiving a dextran sulfate preservative solution in a polycarbonate container, and storing the sample collector in the polycarbonate container with the dextran sulfate preservative solution. The method of collecting the oral fluid sample can include receiving the oral fluid sample with an absorbent pad of the sample collector.

In addition, one embodiment of the claimed invention includes a method of collecting an oral fluid sample that includes viewing an amount of the received oral fluid sample collected through a sample adequacy window in the sample collector. By viewing the amount of sample collected, a user is able to determine sample adequacy volume, that is, whether the volume of sample collected is adequate for testing. Also, the method of collecting an oral fluid sample can include viewing an indicator dye in the amount of the collected oral fluid sample to determine the sample adequacy volume.

In one embodiment, the method of collecting an oral fluid sample includes using a collecting element treated with a hypertonic salt solution including 80 to 170 mg/mL NaCl and 14.3 mg/mL citrate buffer. A flavorant or sweetener can be added to the collecting element as well.

Additionally, in one embodiment of the claimed invention, the method of collecting an oral fluid sample can include using a preservative solution that includes at least one of an anti-microbial agent, an antibacterial agent, an anti-fungal agent, a bacteriostatic agent, a fungistatic agent, and an enzyme inhibitor.

### BRIEF DESCRIPTION OF THE DRAWINGS

FIG. 1 is an exploded view of a sample collector of the claimed invention showing an absorbent pad, an upper casing, and a lower casing.

FIGS. 2A and 2B are top and bottom views, respectively, of a sample collector of the claimed invention showing a collecting element of an absorbent pad and an upper casing with a sample adequacy indicator window.

FIG. 3 is a side view of a sample collector of the claimed invention showing a collecting element of an absorbent pad, an upper casing, and a lower casing.

FIG. 4 is a graph showing increased stability of drugs in a preservative solution of a collecting element in accordance with the claimed invention.

### DETAILED DESCRIPTION

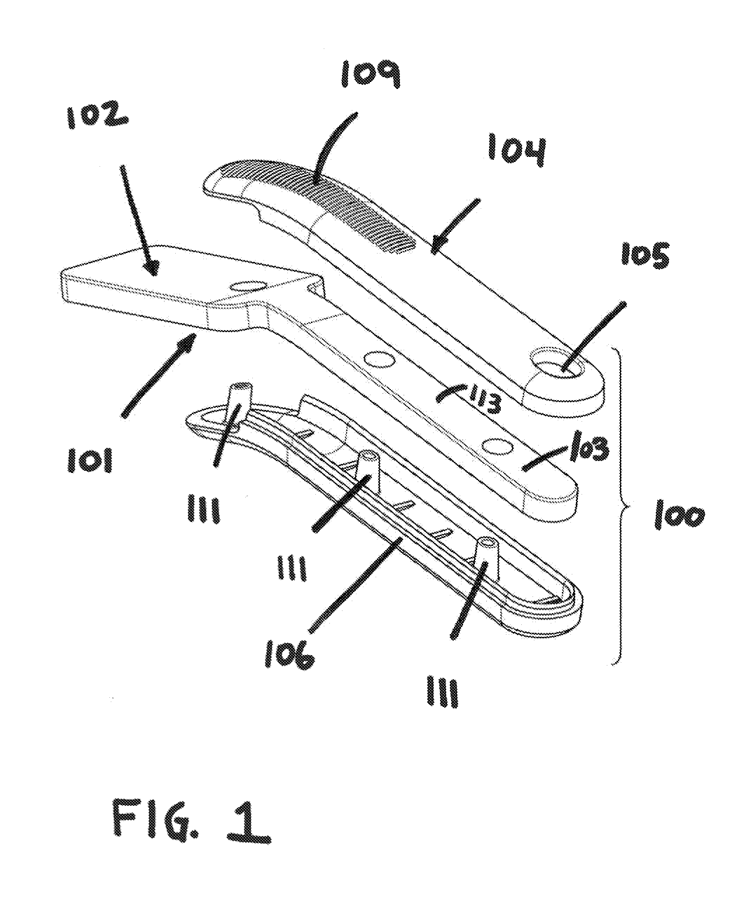

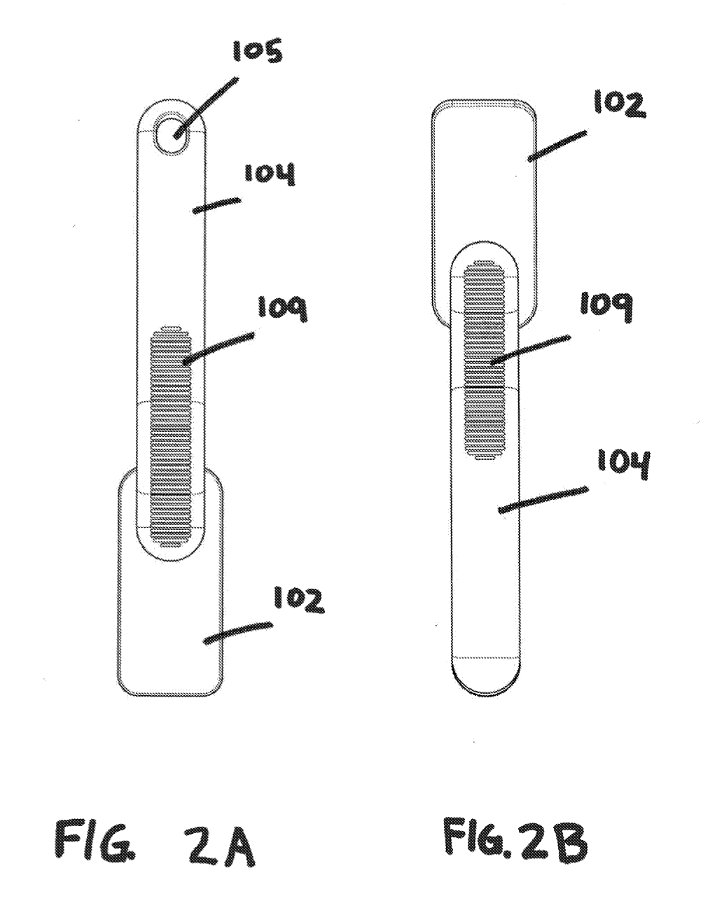

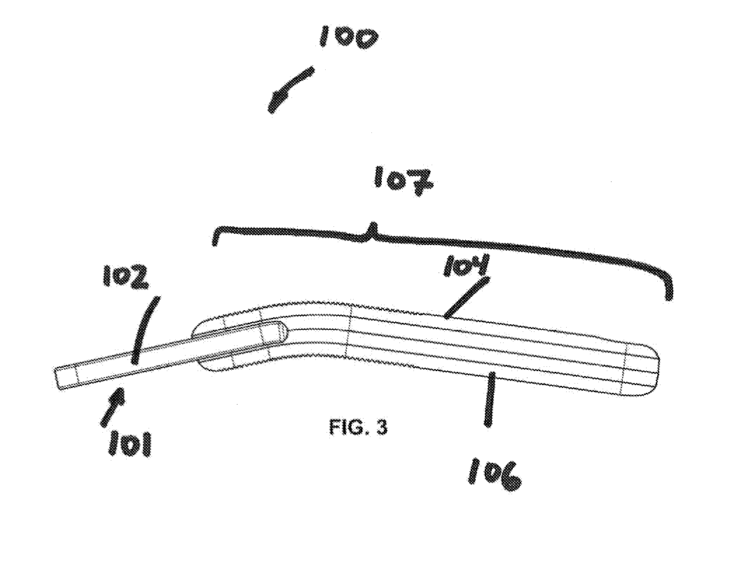

The claimed invention relates to a sample collector 100 for collecting oral fluid samples as shown in FIGS. 1-3. A sample collector 100 has an absorbent pad 101, which has a collecting element 102 and an interior portion 103. The absorbent pad 101 can include an absorbent material including natural occurring absorbent materials such as cotton or cellulose materials as well as synthetic absorbent materials such as, but not limited to, polyesters. As shown in FIGS. 1 and 3, the absorbent pad 101 has a generally flat profile with a collecting element 102, which is shaped to absorb oral fluid when placed within an oral cavity. The collecting element 102 can be any shape such as an oval, a circle, a square, or as shown in FIG. 1, a rectangle. The collecting element 102 can be sized to collect a desired volume of oral fluid. Generally, about 1 mL is collected. When the collecting element 102 is placed into or in contact with the oral cavity of a subject, it absorbs some of the oral fluid from that source. The absorbent pad 101 also had an interior portion 103 connected to the collecting element 102. The interior portion 103 is dimensioned to extend into a handle 107 (shown in FIG. 3) of a sample collector 100 of the invention, which is formed by the upper casing 104 and the lower casing 106. The interior portion 103 extends in the handle 107 past the sample adequacy window 105.

The upper casing 104 and the lower casing 106 are plastic casings which fit together to form the handle 107 of a sample collector of the invention. The handle 107 surrounds the interior portion 103 of the absorbent pad 101. The casings 104 and 106 form a seal when fitted together to form the handle 107. Casings 104 and 106 may be adhered together using a variety of techniques including ultrasonic welding, gluing, and the like. As shown in FIG. 1, the casings 104, 106 can have internal pins 111 to facilitate fitting the upper casing 104 and the lower casing 106 together. Interior portion 103 of the absorbent pad 101 can be shaped to accommodate the fitting mechanism (such as the internal pins 111) of the casings 104, 106. The handle 107 of a sample collector 100 of the claimed invention can be textured to improve handling as indicated by the ridges 109. The sample collector 100 may be straight or angled, as shown in FIGS. 1 and 3.

The upper casing 104 has a sample adequacy window 105 positioned toward the end of the upper casing 104 opposite the collecting element 102. As mentioned above, the interior portion 103 extends in the handle 107 past the sample adequacy window 105. The interior portion 103 is treated with an indicator dye, such as Flag Blue Dye, downstream from the junction of the collecting element 102 and the interior portion 103 but upstream of the area of the interior portion 103 which is seen through the sample adequacy window 105. For example, an indicator dye may be placed about 15 mm upstream from the end of the interior portion 103 at dye placement area 113 as shown in FIG. 1. The dye is placed on the interior portion 103 of the absorbent pad 101 at area 113 and dried before inserting the absorbent pad 101 into the casings 104 and 106 and sealing the device (sample collector 100).

The collecting element 102 can be treated to stimulate oral fluid production and/or to increase the absorbency. For example, absorbent pads treated with a hypertonic salt solution are described in U.S. Pat. No. 5,103,836, which is incorporated by reference in its entirety. To treat the portion of the absorbent pad 101, in one example embodiment, collecting element 102 is allowed to absorb a sufficient amount, approximately 150 uL, of a solution containing 80 to 170 mg/mL (for example, 167 mg/mL) NaCl and 14.3 mg/mL citrate buffer (pH 6.0). Collecting element 102 is then dried. If the treatment causes an unpleasant taste, a flavorant or sweetener or the like can be added to mask the unpleasant taste. Additionally, buffering agents and other agents used to treat oral fluid samples can be dried onto the collecting element 102. The collecting element 102 of the absorbent pad 101 is treated with the solution and then dried before inserting the absorbent pad 101 into the casings 104 and 106 and sealing the device (sample collector 100).

In operation, a user holds a sample collector 100 of the invention by the handle 107 and maneuvers the collecting element 102 of the absorbent pad 101 into or into contact with the subject's oral cavity. The collecting element 102 can be inserted in those areas where oral fluid is excreted and/or collects in the oral cavity. Preferably, the collecting element 102 is placed under the subject's tongue and allowed to collect oral fluid while the device is stationary or the device is moved around the mouth to facilitate the collection. For example, the collecting element 102 may be applied or swabbed inside the mouth, in contact with the gums, to receive a sample of saliva. In another embodiment, the collecting pad 102 is placed inside the mouth between the lower gum and cheek and gently rubbed back and forth along the gum line. In particular, once the collecting element 102 of the absorbent pad 101 comes into contact with an oral fluid source, some of the oral fluid or saliva is drawn, or absorbed, into the absorbent pad 101. The collecting element 102 is left in contact with the oral fluid for a time sufficient to absorb enough oral fluid to fill the absorbent pad 101. As the oral fluid is drawn into and flows into, or wicks up the absorbent pad 101, the fluid encounters an indicator dye dried into the interior portion 103 of the absorbent pad 101 at dye placement area 113. In order to collect a sufficient volume of the sample fluid, the collecting element 102 remains in contact with the oral fluid source until the indicator dye is seen in the sample adequacy window 105. The collecting element 102 may have to remain in contact with the oral fluid source for a specified amount of time. In one example embodiment, the collecting element is placed in contact with the oral fluid for about 30 seconds to about 6 minutes, preferably between about 2 and about 5 minutes. The sample collector 100 is preferably stored in a sealed plastic packaging sleeve and removed just prior to use.

As described previously, samples collected by a sample collector of the invention include saliva, or oral fluid. Accordingly, a further aspect of the invention relates to a method of collecting an oral fluid specimen from an oral cavity for testing. While the method is preferably designed to obtain oral fluid samples to test for drugs of abuse in human subjects, the method can be used to obtain oral fluid sample from humans for other purposes or to obtain oral fluid samples from animals. Once the oral fluid sample is collected, the collecting element 102 is removed from the oral cavity. The fluid sample can then be released, or expressed, from the absorbent pad 101 into a container holding a preservative in a manner employing the systems and devices described above. Alternatively, the collecting element 102 of the sample collector 100 itself can be placed in a preservative solution for later testing of the oral fluid. Thus, it is understood that while the treatments described herein may be employed with the systems and devices described previously, they may be applied more broadly to any system or device for collecting samples of oral fluid.

The oral fluid sample can be expressed from the collection device (sample collector 100) by compressing or squeezing the absorbent pad 101 or by centrifuging the absorbent pad 101. The expressed oral fluid sample can then be analyzed for an analyte of interest. As an alternative to expressing and then analyzing the oral fluid sample, the collecting element 102 containing the oral fluid or the expressed oral fluid sample can also be preserved in a preservative solution for later analysis, as previously described.

A preservative solution, e.g., a preservative solution containing a buffer, surfactant and a salt, can be used with the sample collector of the claimed invention. A preservative solution acts to inhibit enzymatic activity, which can be responsible for the destruction of analytes of interest or can function as an anti-microbial agent. Compounds contemplated for use as a preservative also include antibacterial agents, anti-fungal agents, bacteriostatic agents, fungistatic agents, enzyme inhibitors, and the like.

Table 1 below shows the components and concentration of each component in a preservative used in conjunction with the sample collector 100.

TABLE 1
Preservative Solution

| Component | Amount for 1 L (g) |
|---|---|
| Citric Acid | 0.15 |
| Sodium Citrate Dihydrate | 2.95 |
| Dextran Sulfate Sodium Salt | 0.08 |
| Sodium Benzoate | 2.25 |
| Potassium Sorbate | 2.25 |
| Sodium Chloride | 27.2 |
| ProClin 950 Preservative¹ | 1.05 |
| Tween 20Nonionic Detergent² | 1 |

¹Available from SAFC Supply Solutions, St. Louis, Mo.
²Available from Sigma Aldrich, St. Louis, Mo.

As described previously, samples collected by a sample collector of the invention include saliva, or oral fluid. Accordingly, a further aspect of the invention relates to a method of collecting an oral fluid specimen from an oral cavity for testing. While the method is preferably designed to obtain oral fluid samples to test for drugs of abuse in human subjects, the method may be used to obtain oral fluid sample from humans for other purposes or to obtain oral fluid samples from animals. Once the oral fluid sample is collected, the collecting element 102 is removed from the oral cavity. The fluid sample can then be released, or expressed, from the absorbent material of the absorbent pad 101 into a container containing a preservative in a manner employing the systems and devices described previously. This container and lid containing a preservative is made of polycarbonate plastic Lexan 144R or an equivalent.

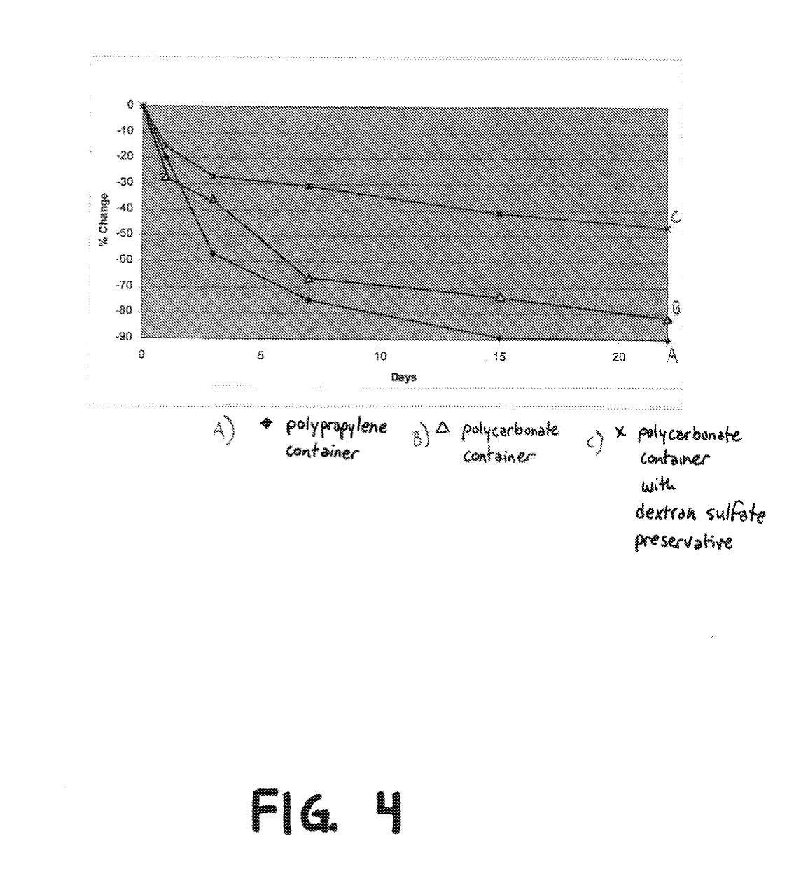

The use of the polycarbonate container and lid and of dextran sulphate as a preservative increases the stability of various drugs in the preservative solution. The graph shown as FIG. 4 illustrates this increased stability for THC when samples are stored at 37° C. Prior devices made of polypropylene contain no dextran sulphate in the preservative solution. This graph of FIG. 4 shows the percent change in sample THC concentration for the sample collection device of the claimed invention in a polypropylene container (filled diamond data points along line A) versus sample THC concentration for a polycarbonate container (open triangle data points along line B) versus sample THC concentration for a polycarbonate container with dextran sulfate preservative (x data points along line C). As shown in the graph, the greatest benefit in maintaining sample concentration is found in using the polycarbonate container with dextran sulfate preservative in accordance with the claimed invention.

That is, the polypropylene container (line A) shows the fastest loss in THC. By changing the polypropylene container to polycarbonate there was a slight increase in THC stability at 15 days from 90% loss to −72% loss. However, the further addition of dextran sulphate to the polycarbonate sample collection device further increased the stability to only a 41% loss at 15 days.

Oral fluid samples collected according to the invention are used in testing for drugs of abuse.

For example, the oral fluid samples can be used to test for marijuana (THC), nicotine (continine), cocaine metabolite (benzoylecgonine), opiates (morphine, 6-acetylmorphine, and codeine), phencyclidine, amphetamines (amphetamine and methamphetamine) and other drugs. A variety of assays and testing methods for such drugs of abuse using oral fluid samples can be used. See, for example, E. J. Cone et al., “Oral Fluid Testing for Drugs of Abuse: Positive Prevalence Rates by Intercept Immunoassay Screening and GC-MS-MS Confirmation and Suggested Cutoff Concentrations,” J. Analytical. Toxicology, vol. 26, p. 541-6, 2002.

Having thus described the basic concept of the invention, it will be rather apparent to those skilled in the art that the foregoing detailed disclosure is intended to be presented by way of example only, and is not limiting. In addition to the embodiments and implementations described above, the invention also relates to the individual components and methods, as well as various combinations and subcombinations within them. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as can be specified in the claims. Accordingly, the invention is limited only by the following claims and equivalents thereto.

The claimed invention is:

1. An oral fluid sample collection kit comprising:
a sample collector including
an absorbent pad with a collecting element and an interior portion;
a handle including an upper casing and a lower casing, the absorbent pad extending into the handle;
a polycarbonate container that receives the absorbent pad; and
a dextran sulfate preservative solution in the polycarbonate container that inhibits enzymatic activity on a collected sample.

2. The oral fluid sample collection kit of claim 1, wherein the handle further comprises:
a sample adequacy window positioned in the handle and providing visual access to the absorbent pad which extends beyond the sample adequacy window.

3. The oral fluid sample collection kit of claim 2, wherein the interior portion of the absorbent pad includes an indicator dye positioned between the junction of the collecting element and the interior portion and the sample adequacy window in the handle.

4. The oral fluid sample collection kit of claim 1, wherein the handle further comprises:
internal pins positioned to fit and secure the upper casing and the lower casing together.

5. The oral fluid sample collection kit of claim 1, wherein the handle further comprises:
textured ridges arranged on at least one of the upper casing and lower casing to provide secure handling of the sample collector.

6. The oral fluid sample collection kit of claim 1, wherein the collecting element includes a hypertonic salt solution including 80 to 170 mg/mL NaCl and 14.3 mg/mL citrate buffer.

7. The oral fluid sample collection kit of claim 6, wherein the collecting element includes a flavorant or sweetener.

8. The oral fluid sample collection kit of claim 1, wherein the preservative solution includes at least one of an anti-microbial agent, an antibacterial agent, an anti-fungal agent, a bacteriostatic agent, a fungistatic agent, and an enzyme inhibitor.

9. A method of collecting an oral fluid sample comprising:
receiving an oral fluid sample with a sample collector;
receiving a dextran sulfate preservative solution in a polycarbonate container; and
storing the sample collector in the polycarbonate container with the dextran sulfate preservative solution.

10. The method of collecting an oral fluid sample of claim 9, wherein receiving the oral fluid sample includes receiving the oral fluid sample with an absorbent pad of the sample collector.

11. The method of collecting an oral fluid sample of claim 9 further comprising:
viewing an amount of the received oral fluid sample collected through a sample adequacy window in the sample collector to determine sample adequacy volume.

12. The method of collecting an oral fluid sample of claim 11 further comprising:
viewing an indicator dye in the amount of the collected oral fluid sample to determine sample adequacy volume.

13. The method of collecting an oral fluid sample of claim 9, wherein the sample collector includes a collecting element treated with a hypertonic salt solution including 80 to 170 mg/mL NaCl and 14.3 mg/mL citrate buffer.

14. The method of collecting an oral fluid sample of claim 9, wherein the collecting element includes a flavorant or sweetener.

15. The method of collecting an oral fluid sample of claim 9, wherein the preservative solution includes at least one of an anti-microbial agent, an antibacterial agent, an anti-fungal agent, a bacteriostatic agent, a fungistatic agent, and an enzyme inhibitor.
